(19) United States
(12) Patent Application Publication
(10) Pub. No.: US 2006/0204550 A1
(43) Pub. Date: Sep. 14, 2006

(54) Food and beverages containing L-carnitine derivatives

(75) Inventor: Kenneth Hassen, (US)

Correspondence Address: OBLON, SPIVAK, MCCLELLAND, MAIER & NEUSTADT, P.C., 1940 DUKE STREET, ALEXANDRIA, VA, 22314, US

(73) Assignee: SIGMA-TAU INDUSTRIE FARMACEUTICHE RIUNITE S.p.A., Rome (IT)

(21) Appl. No.: 11/344,416

(22) Filed: Feb. 1, 2006

Related U.S. Application Data
(60) Provisional application No. 60/660,347, filed on Mar. 11, 2005.

(51) Int. Cl. A61K 47/00; A61K 31/22; A61K 31/205; C07C 229/02

(52) U.S. Cl. 424/439; 514/546; 514/554; 560/155; 562/493

(57) ABSTRACT

The present invention relates to foods and/or. beverages containing L-carnitine derivatives that can be used to supplement L-carnitine deficiencies and/or preserve one or more properties of the foods and/or beverages.

### BACKGROUND OF THE INVENTION

1. Field of the Invention

The present invention relates to foods and/or beverages containing L-carnitine derivatives that can be used to supplement L-carnitine deficiencies and/or preserve one or more properties of the foods and/or beverages.

2. Description of the Background

Increasing awareness to ensure overall good health has brought a new category to the beverage industry called the functional beverage. Functional beverages provide a way to have a more healthy diet. They are also practical because their use does not require a major change in lifestyle. The functional beverage category continues to grow strong in the U.S. market. Functional beverages, ranging from herb-fortified fruit drinks to energy drinks, are now easy to find.

Functional beverages come from the idea that drinks should not only quench thirst but also provide additional health benefits such as to replenish minerals, provide energy, prevent ailments, and promote healthy life styles. These attributes set functional beverages apart from mainstream beverages.

L-carnitine has a physiological role as the carrier of long-chain fatty acid across the internal mitochondrial membrane into the mitochondrial matrix, the site of their oxidation, and since it was first established (Engel and Angelini, Science, 1973, 179: 899-902) that a primary deficiency of L-carnitine is the cause of a severe and sometimes fatal, though rare, form of myopathy (lipid storage myopathy), our knowledge of the pathological consequences of primary and secondary L-carnitine deficiencies and, conversely, of the therapeutic and nutritional value of an exogenous supply of carnitine has increased enormously. Carnitine is present in all biological tissues in relatively high concentrations as free carnitine and in lower concentrations in the form of metabolic products. L-carnitine has also been described to be used in a number of protocols for the treatment of various disorders.

### SUMMARY OF THE INVENTION

Even though any kind of drink can be developed as a functional drink through fortification or ingredient addition, in one embodiment, the invention focuses on the addressing L-carnitine insufficiency in the human diet and for extending the nutritional and flavor attributes of food and beverage platforms, especially the extremely fragile proprietary flavor systems that deliver L-carnitine in such products.

### DETAILED DESCRIPTION OF INVENTION

In one embodiment, the invention provides a salt of L-carnitine or an alkanoyl-L-carnitine with benzoic acid. Examples of alkanoyl-L-carnitines benzoic acid salts include acetyl-L-carnitine benzoic acid salt, propionyl-L-carnitine benzoic acid salt, butyryl-L-carnitine benzoic acid salt, valeryl-L-carnitine benzoic acid salt, and isovaleryl-L-carnitine benzoic acid salt.

In one embodiment, the invention provides a composition suitable for human consumption containing a benzoic acid salt of L-carnitine or an alkanoyl L-carnitine selected from the group consisting of acetyl, propionyl, butyryl, valeryl and isovaleryl, in an amount sufficient to stabilize at least one property of the composition selected from the group consisting of flavor, color, smell and a combination of these; and sufficient to at least partially compensate for a carnitine insufficiency in an individual; and at least one additional edible ingredient.

In certain embodiments, the composition can be in liquid form such as a functional beverage, flavored water, dietary supplement, energy food or a combination of these; or a solid food product. Furthermore, in other embodiments, separately or in combination with the above, the composition can also contain at least one additional source of L-carnitine or a salt thereof such as an alkanoyl derivative of L-carnitine or a salt thereof.

In certain embodiments, separately or in combination with the above, the composition can also contain other edible ingredients such as an electrolyte, a mineral, a vitamin, a coenzyme, an antioxidant, a sweetener, a flavorant, and mixtures thereof.

In one embodiment, the invention provides a packaged material such as a bottle, can or box, containing the composition described herein, which are preferably suitably structured to store and/or deliver the product to the consumer.

In certain embodiments, the composition can be used to treat carnitine deficiency in an individual in need thereof, by providing the composition described herein to the individual whereby the individual consumes at least a part of the composition.

In one embodiment, the invention provides food and/or beverage compositions containing L-carnitine benzoate, and/or any benzoic acid salt of l-carnitine. In such compositions, the treatment or supplementation of carnitine deficiency while simultaneously extending the shelf life of the product into which it is contained can be achieved.

The stable, non-hygroscopic benzoic acid salts of L-carnitine have enhanced nutritional and/or therapeutic efficacy with respect to their inner salts congeners and to the compositions containing the benzoic acid salts of L-carnitine, particularly suited to ingestion in humans.

Carnitine and derivatives of carnitine can be used in various therapeutic uses such as e.g. in the cardiovascular field for the treatment of acute and chronic myocardial ischemia, angina pectoris, heart failure and cardiac arrhythmias. Acetyl L-carnitine is used in the neurological field for the treatment of both central nervous system disturbances and peripheral neuropathies, particularly diabetic peripheral neuropathy. Propionyl L-carnitine is used for the treatment of chronic arteriosclerosis obliterans, particularly in patients showing the symptom of severely disabling intermittent claudication.

In professional athletes and other individuals engaging in physical activity, L-carnitine supplies energy to the skeletal musculature and increases the resistance to prolonged, intense stress, enhancing the performance capability of such individuals.

In addition, the beverages and food compositions described herein can be used as nutritional supplements for both vegetarians, whose diets have a low carnitine content as well as a low content of the two amino acids, lysine and methionine (the precursors of the biosynthesis of L(−)-carnitine in the kidneys and liver) and those subjects who have to live on a diet poor in protein for prolonged periods of time.

Consequently, the beverage and food compositions described herein containing benzoic acid salts of carnitine or derivatives thereof, either as single components or in combinations with further active ingredients, can be in the form of functional beverages, flavored waters, dietary supplements, health foods, energy foods and similar products, such as oleomargarine, butter, cheese, sport supplements, meat replacement products, vegetarian products, baked goods, confectionaries, etc.

Other salts that may be used in place of or in addition to the benzoic acid salts include L-carnitine sorbate (sorbic acid), L-carnitine adipate (adipic acid) and L-carnitine lactate (lactic acid). Each of these salts can perform anti-microbial action within the specific pH range characteristic for meat replacement, vegetarian, bakery and confectionary functional food platforms while providing a source of L-carnitine supplementation. In addition, other carnitine derivatives, such as salt forms and alkanoyl derivatives may be included in the compositions described herein.

Many fruit flavors, herbs, sweeteners, level of sweetness, vitamins, and nutritional supplements can be chosen, and therefore may combinations and products are possible. Packaging design sometimes can serve as a good product differentiator. Therefore, in one embodiment, an article such as a bottle, can or box containing the compositions of the invention is provided. The article can be composed of food-grade materials such as plastics, paper, aluminum and others. In a preferred embodiment, the article is configured in such a way to permit storage and/or delivery of the composition to the consumer.

The compositions can be provided as dry powders by freeze drying, spray drying, etc, and as liquids, each of which may be premixed to deliver ready-to-use composition or as a concentrate which can be incorporated into liquid and/or solid edible mediums. If liquid, the composition can be carbonated or still. The compositions of the present invention can be provided as a variety of forms of .foods and beverages, for example, beverages such as sport drinks, fruit beverages, tea beverages, vegetable juices, dairy beverages, milk beverages, alcoholic drinks, jelly drinks, carbonated drinks, and the like; confectionaries such as jelly, chewing gum, chocolate, ice cream, candy, biscuit, or the like; starch-based processed foods such as snacks, breads, cakes, or the like; protein-based processed foods such as. meat or meat substitute products, cheese, etc; and supplements.

Additional additives to the compositions described herein include carbohydrates, electrolytes, carotenoids, vitamins, minerals, antioxidants, proteins, amino acids such as taurine, glutamine, and arginine, sugar or other natural or artificial sweeteners such as sucralose, aspartame, acesulfame-K, saccharin, cyclamate, neohesperidin dihydrochalcone, thaumatin, altame, stevioside, and glycyrrhzin, caffeine, ginseng, natural lipids and oils, plant extracts, fiber, fruits, jerry, or the like, milk components, flavors, flavor enhancers, food-approved colors, glucides such as flour, starches, hormones, nourishing components, and the like.

The flavors useful in the compositions described herein are sometimes available as dry ingredients, liquids or emulsions. They can be mixed into the concentrate or final product form. In addition to the active flavor itself, industrially available flavors can contain, weighting agents, emulsifiers, emulsion stabilizers, antioxidants, liquid vehicles, and other common beverage and food manufacturing ingredients.

The particular amount of the flavor component effective for imparting flavor characteristics depend upon the flavor, the flavor impression and formula flavor component. For example, using beverages as an example, the flavor can be included in at least 0.05% by weight of the beverage composition and, for example, in a range of from 0.1% to about 2% by weight of the beverage in its final ready-to-use stage. In addition to fruity flavors, such as lemon, orange, lime, cherry, strawberry, raspberry, grape, grapefruit, and others, other flavors, e.g. cola, root beer, coffee, tea, botanical flavors can also be included in the compositions as well as those derived from nuts, bark, roots and leaves. Combinations are may also be used.

The ideal combination of color, taste, consistency (mouth feel) and appearance can pose shelf stability problems. A citrus green or carrot orange beverage will look best on the shelf if packaged in a clear container. Clear, light admitting containers, along with the aqueous environment and contained sweeteners may significantly contribute to fermentative and oxidative reactions that at least tend to rapidly destroy the delicate flavor attributes of these products. Flavor systems, critical to both brand development and repeat sales; tend to be the most expensive part of any beverage formulation.

The addition of benzoic acid salts of L-carnitine can improve shelf life for these types of products relative to a similar product not containing the benzoic acid salt of L-carnitine. Food grade benzoic acid, itself difficult to handle for reasons of dissolution, performs relatively well in a reduced sodium or potassium salt. Such preservatives have wide acceptance in the beverage industry, since they maintain a consistently acidic pH that naturally inhibits mold and other microbial growth of the type that disturbs beverage gas content and destroys flavor attributes. This is typically accomplished at concentrations of 0.1% on a w/v basis. This can permit the preparation of ready to mix flavor bases that may be economically shipped to distant markets, an additional benefit.

L-Carnitine inner salt can be made in an acid base reaction with molten benzoic acid in such a way as to produce the a salt solution that may be dried to a fine white powder within a gas fired, rotary drum or similar dryer to a water content of LT 2%. It may then be further processed, e.g., drummed, or entered in various forms of roller compaction (Chilsonation) then milled and screened to obtain preferred particle sizes. In one embodiment, particle size would be 100% to pass a number 100 USBS mesh screen. Particles may then be blended with a food grade, hydrophobic fumed or precipitated silica(s) that possesses an overall surface area of 190-475 square meters per gram and a tapped density of 80-275 grams per liter.

Obviously, numerous modifications and variations of the present invention are possible in light of the above teachings. It is therefore to be understood that within the scope of the appended claims, the invention may be practiced otherwise than as specifically described herein.

1. A salt of L-carnitine or alkanoyl-L-carnitine with benzoic acid.

2. The salt of claim 1, wherein the alkanoyl is selected from the group consisting of acetyl, propionyl, butyryl, valeryl and isovaleryl.

3. L-carnitine benzoate.

4. Acetyl L-carnitine benzoate.

5. Propionyl L-carnitine benzoate.

6. A composition comprising as an active ingredient at least one salt according to claim 1; and at least one additional ingredient.

7. The composition of claim 6 suitable for human consumption, comprising
a benzoic acid salt of carnitine in an amount sufficient to stabilize at least one property of the composition selected from the group consisting of flavor, color, smell and a combination of these; and sufficient to at least partially compensate for a carnitine insufficiency in an individual; and
at least one additional edible ingredient.

8. The composition of claim 7, which is in liquid form.

9. The composition of claim 8, wherein the composition is a functional beverage, flavored water, dietary supplement, energy food or a combination of these.

10. The composition of claim 7, which is a solid food product.

11. The composition of claim 7, further comprising at least one additional source of L-carnitine or a salt thereof.

12. The composition of claim 7, wherein the at least one additional source of L-carnitine is an alkanoyl derivative of L-carnitine or a salt thereof.

13. The composition of claim 7, wherein the at least one edible ingredient is selected from the group consisting of an electrolyte, a mineral, a vitamin, a coenzyme, an antioxidant, a sweetener, a flavorant, and mixtures thereof.

14. A packaged material comprising the composition of claim 7.

15. A packaged material comprising the composition of claim 8.

16. A packaged material comprising the composition of claim 9.

17. A packaged material comprising the composition of claim 10.

18. A packaged material comprising the composition of claim 11.

19. A packaged material comprising the composition of claim 12.

20. A packaged material comprising the composition of claim 13.

21. A method of treating a carnitine deficiency in an individual in need thereof, comprising providing the composition of claim 7 to the individual whereby the individual consumes at least a part of the composition thereby treating the carnitine deficiency in the individual.
